(19) United States
(12) Patent Application Publication
(10) Pub. No.: US 2004/0023951 A1
(43) Pub. Date: Feb. 5, 2004

(54) Combination therapy for treatment of psychoses

(76) Inventors: Franklin Porter Bymaster, Brownsburg, IN (US); Harlan Edgar Shannon, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/296,738

(22) PCT Filed: Jun. 18, 2001
(86) PCT No.: PCT/US01/14863

(51) Int. Cl. A61K 31/551; A61K 31/4439

(52) U.S. Cl. 514/220; 514/342

(57) ABSTRACT

The invention provides combination therapy comprising a first component which is a typical antipsychotic or an atypical antipsychotic and a second component which is a muscarinic agonist for the treatment of psychoses and other disorders.

The present invention belongs to the fields of pharmacology, medicine, and medicinal chemistry, and provides methods and compositions for the treatment of a disorders, including psychoses.

### BACKGROUND OF THE INVENTION

Psychoses are serious mental illnesses characterized by defective reality or lost contact with reality. Psychotic patients may suffer hallucinations, delusions, and grossly disorganized behavior as part of their disease. Psychoses exact a tremendous emotional and economic toll on the patients, their families, and society as a whole. For example, it has been estimated that as many as 50% of the homeless people living in the United States are psychotic. (Bachrach, Treating the Homeless Mentally Ill, Washington, D.C., American Psychiatric Press, 1340, Lamb et al. ed. (1992)). In addition, approximately 2.5% of the total dollars spent for health care in the United States is spent in the treatment of psychoses (Rupp et al., Psychiatric Clin. North Am., 16:413-423 (1993)).

Several classes of compounds are useful for treating psychotic disorders. The drugs available for such conditions are often associated with undesirable side effects. Furthermore, many patients do not respond or only partially respond to present drug treatment, and estimates of such partial-responders and non-responders vary between 40% and 80% of those treated. Thus, further methods of treating psychoses are highly desirable.

The present invention addresses these needs by providing a method of treating psychosis, and other disorders as described herein, by the synergistic effect of combination therapy of a typical or atypical antipsychotic and a muscarinic agonist.

### SUMMARY OF THE INVENTION

The invention provides a method for treating a patient suffering from or susceptible to psychosis, comprising administering to the patient an effective amount a first component which is a typical antipsychotic or an atypical antipsychotic and an effective amount of a second component which is a muscarinic agonist.

The invention also provides a pharmaceutical composition which comprises a first component which is a typical antipsychotic or an atypical antipsychotic, and a second component which is a muscarinic agonist.

That is, the present invention provides the use of a pharmaceutical composition comprising an effective amount of a first component which is a typical antipsychotic or an atypical antipsychotic, in combination with an effective amount of a second component which is a muscarinic agonist for treating psychosis.

### DETAILED DESCRIPTION OF THE INVENTION

The present invention provides for treatment for patients suffering from psychosis comprising the administration of a typical antipsychotic or an atypical antipsychotic and a muscarinic agonist. It has been discovered that the administration of a typical antipsychotic or an atypical antipsychotic and a muscarinic agonist unexpectedly enhances the therapeutic effect of the combination. That is, the combined administration of a typical antipsychotic or an atypical antipsychotic and a muscarinic agonist provides a synergistic effect. Thus, the combination therapy of the present invention provides an effective treatment of psychoses with lessened side effects and of broader applicability than each of the individual components alone.

Within the context of the present invention, the term “psychosis” includes schizophrenia, schizophreniform diseases, mania, schizoaffective disorders, and depression with psychotic features. The above mentioned conditions represent multiple disease states. For example, schizophrenia is referred to in various forms as catatonic, disorganized, paranoid, undifferential, residual, among others. All the various forms of the disorders mentioned herein are contemplated as part of the present invention.

The following list further illustrates a number of these disease states, many of which are classified in the Diagnostic and Statistical Manual of Mental Disorders, 4th Edition, published by the American Psychiatric Association (DSM IV): Paranoid Type Schizophrenia, Disorganized Type Schizophrenia, Catatonic Type Schizophrenia, Undifferentiated Type Schizophrenia, Residual Type Schizophrenia, Schizophreniform Disorder, Schizoaffective Disorder, Delusional Disorder, Brief Psychotic Disorder, Shared Psychotic Disorder, Psychotic Disorder Due to a General Medical Condition, Substance-Induced Psychotic Disorder, Psychotic Disorder Not Otherwise Specified, Major Depressive Disorder with Psychotic Features, Bipolar Disorder I, Bipolar Disorder II, Bipolar Disorder Not Otherwise Specified, Schizoid Personality Disorder, and Schitzotypal Personality Disorder.

In addition, other disorders that are treated by the present combination include, dementia, including Alzheimer's disease, mood disorders, including depression, anxiety disorders, including general anxiety disorder and panic disorder, adjustment disorders, decreased cognition.

All of these disorders are readily diagnosed by the skilled clinician using well established criteria, including those in the DSM IV. In particular, a patient suffering from or susceptible to psychosis can be readily diagnosed using the methods described in the DSM-IV and other criteria known in the art.

As will be appreciated by the skilled person, there are alternative nomenclatures, nosologies, and classification systems for the psychoses described herein and that these systems evolve with medical scientific progress. Applicants do not intend that the present invention be limited to any disorders literally mentioned in the DSM-IV.

The term “patient” refers to a mammal and includes, mice, rats, dogs, sheep, cattle, pigs, guinea pigs, cats, chimpanzees, monkeys, apes, and humans, etc. In particular the term includes a human suffering from psychosis.

The term “effective amount of a first component” and “an effective amount a second component” refers to the amounts of typical antipsychotic or an atypical antipsychotic and amounts of a muscarinic agonist, respectively, which, upon single or multiple administration to the patient, is synergistically effective in alleviating or controlling the disorders described herein, and in particular psychosis.

An effective amount of a first component and an effective amount of a second component can be readily determined by the attending diagnostician, as one skilled in the art, by the use of conventional techniques and by observing results. In determining an effective amount of the first and second components a number of factors are considered by the attending diagnostician, including, but not limited to: the properties of the individual components, the properties of the components in combination as determined in preclinical and clinical trials, the dose of each component, the species of mammal; its size, age, and general health; the specific disorder(s) involved; the degree of involvement or the severity of the disorder(s); the response of the individual patient; the mode of administration; the bioavailability of the formulation administered; the dose regimen selected; and other factors known in the art.

Some preferred dosages are provided here for dosing of the combination of xanomeline and olanzapine: xanomeline, from about 1 to 225 mg per day, preferred; and most preferably about 25 to 125 mg per day; and for olanzapine from about 0.25 to 50 mg, once/day; preferred, from 1 to 30 mg, once/day; and most preferably 1 to 25 mg once/day.

The class of compounds referred to a “typical antipsychotic” are effective in improving symptoms of psychoses, and in particular schizophrenia, by acting as dopamine receptor antagonists, more particularly D₂ dopamine receptor antagonists, which also are known as D₂ dopamine receptor blockading agents. As used herein, the term “typical antipsychotic” include those drugs known as typical neuroleptics.

As used herein the term “typical antipsychotic” includes, but is not limited to, thiopropazate, chlorpromazine, triflupromazine, mesoridazine, piperacetazine, thioridazine, acetophenazine, fluphenazine, perphenazine, trifluoperazine, chlorprathixene, thiothixene, haloperidol, bromperidol, loxapine, molindone, loxapine, molindone, and pimozide. These compound are well known in the art.

The class of compounds referred to a “atypical antipsychotic” are effective in improving symptoms of psychoses, and in particular schizophrenia. These compounds act be a variety of mechanisms, including antagonism of D₂, D₃ and D₄ dopamine neurons, 5-HT₂ receptors, and an alpha₂-adrenergic receptors. As used herein, the term “atypical antipsychotic” include those drugs known as atypical neuroleptics.

As used herein the term “atypical antipsychotic” includes, but is not limited to, clozapine, 8-chloro-11-(4-methyl-1-piperazinyl)-5H-dibenzo[b,e][1,4]diazepine (U.S. Pat. No. 3,539,573); olanzapine, 2-methyl-4-(4-methyl-1-piperazinyl)-10H-thieno[2,3-b][1,5]benzodiazepine (U.S. Pat. No. 5,229,382); zotepine, iloperidone, amisulpiride perospirone, risperidone, 3-[2-[4-(6-fluoro-1,2-benzisoxazol-3-yl)piperidino]ethyl]-2-methyl-6,7,8,9-tetrahydro-4H-pyrido[1,2-a]pyrimidin-4-one (U.S. Pat. No. 4,804,663); and sertindole, 1-[2-[4-[5-chloro-1-(4-fluorophenyl)-1H-indol-3-yl]-1-piperidinyl]ethyl]imidazolidin-2-one (U.S. Pat. Nos. 4,710,500; 5,112,838; 5,238,945); quetiapine, 5-[2-(4-dibenzo[b,f][1,4]thiazepin-11-yl-1-piperazinyl)ethoxy]ethanol (U.S. Pat. No. 4,879,288); and ziprasidone, 5-[2-[4-(1,2-benzoisothiazol-3-yl)-1-piperazinyl]ethyl]-6-chloro-1,3-dihydro-2H-indol-2-one (U.S. Pat. Nos. 4,831,031 and 5,312,925). These compound are also well known in the art.

The class of compounds referred to as “muscarinic agonists” are effective in improving symptoms of psychoses, and in particular schizophrenia. As used herein the term “muscarinic agonist” includes, but is not limited to, pilocarpine, oxotremorine, bethanechol, carachol, sabcomeline, milameline, talsaclidine, and the compounds of the formula I, II, and III below:

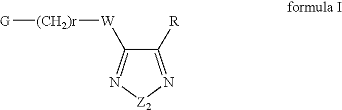

wherein

W is oxygen or sulfur;

R is selected from the group consisting of —OR⁴, —SR⁴, —Z—C₃₋₁₀-cycloalkyl and —Z—C₄₋₁₂-(cycloalkylalkyl);

R⁴ is selected from the group consisting of C₁₋₁₅-alkyl, C₂₋₁₅-alkenyl and C₂₋₁₅-alkynyl, each of which is optionally substituted with one or more independently selected from the group consisting of halogen(s), —CF₃, —CN, C₁₋₄-alkoxy, phenyl, and phenoxy wherein the phenyl or phenoxy is optionally substituted with one or more independently selected from the group consisting of halogen, —CN, C₁₋₄-alkyl, C₁₋₄-alkoxy, —OCF₃, —CF₃, —CONH₂ and —CSNH₂;

Z is oxygen or sulphur;

Z² is oxygen or sulphur

G is selected from the group consisting of:

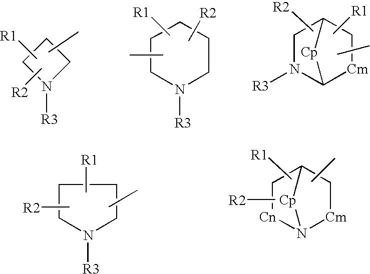

R¹ and R² independently are selected from the group consisting of hydrogen, C₁₋₁₅-alkyl, C₂₋₅-alkenyl, C₂₋₅-alkynyl, C₁₋₁₀-alkoxy, and C₁₋₅-alkyl substituted with one or more independently selected from the group consisting of —OH, —COR⁶, CH₂—OH, halogen, —NH₂, carboxy, and phenyl;

R⁶ is hydrogen, C₁₋₆-alkyl;

R³ is selected from the group consisting of hydrogen, C₁₋₅-alkyl, C₂₋₅-alkenyl and C₂₋₅-alkynyl;

n is 1 or 2;

m is 1 or 2;

p is 1 or 2;

q is 1 or 2;

r is 0, 1 or 2; or

a pharmaceutically acceptable salt thereof;

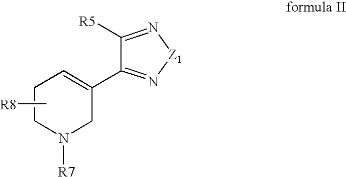

R5 is selected from the group consisting of —OR⁴, —SR⁴, R⁴ is selected from the group consisting of C₁₋₁₅-alkyl, C₂₋₁₅-alkenyl and C₂₋₁₅-alkynyl, each of which is optionally substituted with one or more independently selected from the group consisting of halogen(s), —CF₃, —CN, C₁₋₄-alkoxy, phenyl, and phenoxy wherein the phenyl or phenoxy is optionally substituted with one or more independently selected from the group consisting of halogen, —CN, C₁₋₄-alkyl, C₁₋₄-alkoxy, —OCF₃, —CF₃, —CONH₂ and —CSNH₂;

Z₁ is oxygen or sulphur,

R7 is selected from the group consisting of hydrogen, C₁₋₁₅-alkyl, C₂₋₅-alkenyl, C₂₋₅-alkynyl;

R8 is selected from the group consisting of hydrogen, and C₁₋₄-alkyl; or

a pharmaceutically acceptable salt thereof;

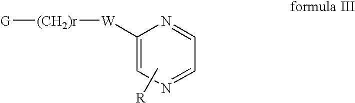

W is oxygen or sulfur;

R is selected from the group consisting of —OR⁴, —SR⁴, —Z—C₃₋₁₀-cycloalkyl and —Z—C₄₋₁₂-(cycloalkylalkyl);

R⁴ is selected from the group consisting of C₁₋₁₅-alkyl, C₂₋₁₅-alkenyl and C₂₋₁₅-alkynyl, each of which is optionally substituted with one or more independently selected from the group consisting of halogen(s), —CF₃, —CN, C₁₋₄-alkoxy, phenyl, and phenoxy wherein the phenyl or phenoxy is optionally substituted with one or more independently selected from the group consisting of halogen, —CN, C₁₋₄-alkyl, C₁₋₄-alkoxy, —OCF₃, —CF₃, —CONH₂ and —CSNH₂;

Z is oxygen or sulphur,

G is selected from the group consisting of:

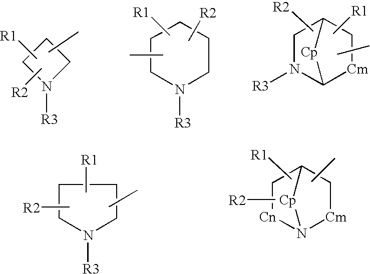

R¹ and R² independently are selected from the group consisting of hydrogen, C₁₋₁₅-alkyl, C₂₋₅-alkenyl, C₂₋₅-alkynyl, C₁₋₁₀-alkoxy, and C₁₋₅-alkyl substituted with one or more independently selected from the group consisting of —OH, —COR⁶, CH₂—OH, halogen, —NH₂, carboxy, and phenyl;

R⁶ is hydrogen, C₁₋₆-alkyl;

R³ is selected from the group consisting of hydrogen, C₁₋₅-alkyl, C₂₋₅-alkenyl and C₂₋₅-alkynyl;

n is 1 or 2;

m is 1 or 2;

p is 1 or 2;

q is 1 or 2;

r is 0, 1 or 2; or

a pharmaceutically acceptable salt thereof.

The compounds of formula I, II, and III are readily prepare according to the methods described in U.S. Pat. Nos. 5,043,345; 5,968,926; 5,744,489; and 5,998,404.

Preferred compounds of formula I are (±)3-{3-[4-(trifluoromethyl)phenyl]propylthio}-4-[-3-(1-azabicyclo[2.2.2]octyloxy)]-1,2,5thiadiazole, (±)-3-Methoxy-4-(1-azabicyclo[2.2.2]octyl-3-oxy)-1,2,5-thiadiazole, (±)-3-Ethoxy-4-(1-azabicyclo[2.2.2]octyl-3-oxy)-1,2,5-thiadiazole, (±)-3-Propyloxy-4-(1-azabicyclo[2.2.2]octyl-3-oxy)-1,2,5-thiadiazole, (±)-3-Butyloxy-4-(1-azabicyclo[2.2.2]octyl-3-oxy)-1,2,5thiadiazole, (±)-3-Pentyloxy-4-(1-azabicyclo[2.2.2]octyl-3-oxy)-1,2,5-thiadiazole, (±)-3-Hexyloxy-4-(1-azabicyclo[2.2.2]octyl-3-oxy)-1,2,5-thiadiazole, (±)-3-(4-Methylpentyloxy)-4-(1-azabicyclo[2.2.2]octyl-3-oxy)-1,2,5-thiadiazole, (±)-3-Propylthio-4-(1-azabicyclo[2.2.2]octyl-3-oxy)-1,2,5-thiadiazole, (±)-3-Butylthio-4-(1-azabicyclo[2.2.2]octyl-3-oxy)-1,2,5-thiadiazole, (±)-3-Pentylthio-4-(1-azabicyclo[2.2.2]octyl-3-oxy)-1,2,5-thiadiazole, (S)-3-Pentylthio-4-(1-azabicyclo[2.2.2]octyl-3-oxy)-1,2,5-oxadiazole,

(±)-3-Hexylthio-4-(1-azabicyclo[2.2.2]octyl-3-oxy)-1,2,5-thiadiazole, (±)-3-(2,2,3,3,3-Pentafluoropropylthio)-4-(1-azabicyclo[2.2.2]octyl-3-oxy)-1,2,5-thiadiazole, (±)-3-Butylthio-4-((1-azabicyclo[2.2.2]octan-3-yl)methoxy)-1,2,5-thiadiazole, (±)-Exo-3-pentylthio-4-(1-azabicyclo[3.2.1]octyl-6-oxy)-1,2,5-thiadiazole, (±)-Endo-3-pentylthio-4-(1-azabicyclo[3.2.1]octyl-6-oxy)-1,2,5-thiadiazole, (±)-Endo-3-butyloxy-4-(1-azabicyclo[2.2.1]heptyl-3-oxy)-1,2,5-thiadiazole, (±)-Exo-3-butyloxy-4-(1-azabicyclo[2.2.1]heptyl-3-oxy)-1,2,5-thiadiazole, (±)-3-Butyloxy-4-(3-pyrrolidinyloxy)-1,2,5-thiadiazole, (±)-3-Butyloxy-4-(1-methyl-3-pyrrolidinyloxy)-1,2,5-thiadiazole, (±)-3-Butylthio-4-(1-methyl-3-piperidyloxy)-1,2,5-thiadiazole, 3-Butylthio-4-(1-methyl-4-piperidyloxy)-1,2,5-thiadiazole, (S)-3-Butyloxy-4-(1-methyl-2-pyrrolidinylmethoxy)-1,2,5-thiadiazole, (S)-3-Butyloxy-4-(2-pyrrolidinylmethoxy)-1,2,5-thiadiazole, (R)-3-Pentylthio-4-(1-azabicyclo[2.2.2]octyl-3-oxy)-1,2,5-thiadiazole, (±)-3-(4-Methylpentylthio)-4-(1-azabicyclo[2.2.2]octyl-3-oxy)-1,2,5-thiadiazole, (±)-3-(3-Phenylpropylthio)-4-(1-azabicyclo[2.2.2]octyl-3-oxy)-1,2,5-thiadiazole, (±)-3(4-Cyanobenzylthio)-4-(1-azabicyclo[2.2.2]octyl-3-oxy)-1,2,5-thiadiazole, (±)-3-(4-Fluorobenzylthio)-4-(1-azabicyclo[2.2.2]octyl-3-oxy)-1,2,5-thiadiazole, (±)-3-(2-Phenylethylthio)-4-(1-azabicyclo[2.2.2]octyl-3-oxy)-1,2,5-thiadiazole, (±)-3-(2-Phenyloxyethylthio)-4-(1-azabicyclo[2.2.2]octyl-3-oxy)-1,2,5-thiadiazole, (±)-Endo-3-(4-cyanobenzylthio)-4-(1-azabicyclo[3.2.1]octyl-6-oxy)-1,2,5-thiadiazole, 3-Butyloxy-4-(3-azetidinyloxy)-1,2,5-thiadiazole, 3-Butylthio-4-(3-azetidinyloxy)-1,2,5-thiadiazole, (±)-3-Butylthio-4-(3-pyrrolidinyloxy)-1,2,5-thiadiazole, (+/−)-3-butylthio-4-(azabicyclo[2.2.2]octyl-3-oxy)-1,2,5-oxadiazole, (+/−)-3-(2-butyloxy)-4-[(+/−)-3-azabicyclo[2.2.2]octyloxy)-1,2,5-oxadiazole, 3-(2,2,3,3,4,4,4-heptaflurorobutyloxy)-4-[(+/−)-3-(1-azabicyclo[2.2.2]octyloxy)]-1,2,5-oxadiazole, 3-methoxy-4-(1-azabicyclo[2.2.2]octyl-3-oxy)-1,2,5-oxadiazole, 3-pentylthio-4-(1-azabicyclo[2.2.2]ocytl-3-oxy)-1,2,5-oxadiazole, trans-3-butyloxy-4-(2-dimethylaminocyclopentyloxy)-1,2,5-oxadiazole, 3-butylthio-4-(3-azetidinyloxy)-1,2,5-oxadiazole, 3-(3-N-(2-thiazolidonyl)propylthio)-4-(1-azabicyclo[2.2.2]octyl-3-oxy)-1,2,5-oxadiazole, 3-chloro-4-(1-azabicyclo[3.2.1]octyl-6-oxy)-1,2,5-oxadiazole and (+/−)-3-butyloxy-4-[endo-(+/−)-6-[1-azabicyclo[3.2.1]octyloxy)]-1,2,5-oxadiazole.

Particularly preferred compounds of formula II are 1,2,5,6-Tetrahydro-3-(3-methoxy-1,2,5-thiadiazol-4-yl)-1-methylpyridine; 3-(3-Ethoxy-1,2,5-thiadiazol-4-yl)-1,2,5,6-tetrahydro-1-methylpyridine; 1,2,5,6-Tetrahydro-1-methyl-3-(3-propoxy-1,2,5-thiadiazol-4-yl)pyridine; 3-(3-Butoxy-1,2,5-thiadiazol-4-yl)-1,2,5,6-tetrahydro-1-methylpyridine; 1,2,5,6-Tetrahydro-3-(3-isopropoxy-1,2,5-thiadiazol-4-yl)-1-methylpyridine; 1,2,5,6-Tetrahydro-1-methyl-3-(3-pentyloxy-1,2,5-thiadiazol-4-yl)pyridine; 1,2,5,6-Tetrahydro-3-(3-isobutoxy-1,2,5-thiadiazol-4-yl)-1-methylpyridine; 1,2,5,6-Tetrahydro-3-(3-isopentyloxy-1,2,5-thiadiazol-4-yl)-1-methylpyridine; 3-(3-Hexyloxy-1,2,5-thiadiazol-4-yl)-1,2,5,6-tetrahydro-1-methylpyridine; 3-(3-(3-Butenyloxy)-1,2,5-thiadiazol-4-yl)-1,2,5,6-tetrahydro-1-methylpyridin e; 3-(3-(2-Butynyloxy)-1,2,5-thiadiazol-4-yl)-1,2,5,6-tetrahydro-1-methylpyridine; 1,2,5,6-Tetrahydro-1-methyl-3-(3-propargyloxy-1,2,5-thiadiazol-4-yl)pyridine; 3-(3-Butoxy-1,2,5-thiadiazol-4-yl)-1,2,5,6-tetrahydropyridine; 3-(3-Ethoxy-1,2,5-thiadiazol-4-yl)-1-ethyl-1,2,5,6-tetrahydropyridine; 3-(3-Heptyloxy-1,2,5-thiadiazol-4-yl)-1,2,5,6-tetrahydro-1-methylpyridine; 3-(3-(3-Pentynyloxy)-1,2,5-thiadiazol-4-yl)-1,2,5,6-tetrahydro-1-methylpyri dine; 3-(3-(4-Pentenyloxy)-1,2,5-thiadiazol-4-yl)-1,2,5,6-tetrahydro-1-methylpyri dine; 3-(3-(2-Propenyloxy)-1,2,5-thiadiazol-4-yl)-1,2,5,6-tetrahydro-1-methylpyri dine; 3-(3-Octyloxy-1,2,5-thiadiazol-4-yl)-1,2,5,6-tetrahydro-1-methylpyridine; 3-(3-(3-Hexynyloxy)-1,2,5-thiadiazol-4-yl)-1,2,5,6-tetrahydro-1-methylpyridine; 3-(3-(3-Methyl-2-butenyloxy)-1,2,5-thiadiazol-4-yl)-1,2,5,6-tetrahydro-1-methylpyridine; 3-(3-(3-Butenyl-2-oxy)-1,2,5-thiadiazol-4-yl)-1,2,5,6-tetrahydro-1-methylpyridine; 3-(3-(4-Hexenyloxy)-1,2,5-thiadiazol-4-yl)-1,2,5,6-tetrahydro-1-methylpyridine; trans-3-(3-(3-Hexenyloxy)-1,2,5-thiadiazol-4-yl)-1,2,5,6-tetrahydro-1-methy-1-pyridine; cis-3-(3-(2-Pentenyloxy)-1,2,5-thiadiazol-4-yl)-1,2,5,6-tetrahydro-1-methylpyridine; cis-3-(3-(2-Hexenyloxy)-1,2,5-thiadiazol-4-yl)-1,2,5,6-tetrahydro-1-methylpyridine; 3-(3-(5-Hexenyloxy)-1,2,5-thiadiazol-4-yl)-1,2,5,6-tetrahydro-1-methylpyridine; cis-3-(3-(3-Hexenyloxy)-1,2,5-thiadiazol-4-yl)-1,2,5,6-tetrahydro-1-methylpyridine; trans-3-(3-(2-Hexenyloxy)-1,2,5-thiadiazol-4-yl)-1,2,5,6-tetrahydro-1-methy-1-pyridine; ,2,5,6-Tetrahydro-3-(3-hexyloxy-1,2,5-thiadiazol-4-yl)pyridine; 1,2,5,6-Tetrahydro-3-(3-methoxy-1,2,5-thiadiazol-4-yl)-1,4-dimethylpyridine; 3-(3-Hexyloxy-1,2,5-thiadiazol-4-yl)-1,2,5,6-tetrahydro-1,4-dimethylpyridine; 3-(3-Hexyloxy-1,2,5-thiadiazol-4-yl)-1,2,5,6-tetrahydro-1,6-dimethylpyridine; 3-(3-Pentyloxy-1,2,5-thiadiazol-4-yl)-1,2,5,6-tetrahydro-1,6-dimethylpyridine; 3-(3-Butoxy-1,2,5-thiadiazol-4-yl)-1,2,5,6-tetrahydro-1,6-dimethylpyridine; 3-(3-(4-Pentenyloxy)-1,2,5-thiadiazol-4-yl)-1,2,5,6-tetrahydro-1,6-dimethylpyridine; 3-(3-(3-Hexynyloxy)-1,2,5-thiadiazol-4-yl)-1,2,5,6-tetrahydro-1,6dimethylpyridine; 3-(3-Ethoxy-1,2,5-thiadiazol-4-yl)-1,2,5,6-tetrahydro-1,6-dimethylpyridine; 3-(3-(1-Ethylpentyloxy)-1,2,5-thiadiazol-4-yl)-1,2,5,6-tetrahydro-1-methylpyridine; 3-(3-(1-Ethylbutoxy)-1,2,5-thiadiazol-4-yl)-1,2,5,6tetrahydro-1-methylpyri dine; 3-(3-(1-Methylpentyloxy)-1,2,5-thiadiazol-4-yl)-1,2,5,6-tetrahydro-1-methylpyridine; 3-(3-(5-Hexynyloxy)-1,2,5-thiadiazol-4-yl)-1,2,5,6-tetrahydro-1-methylpyridine; 3-(3-(3-Methyl-4-pentenyloxy)-1,2,5-thiadiazol-4-yl)-1,2,5,6-tetrahydro-1-methylpyridine; 3-(3-(2,3-Dimethylpentyloxy)-1,2,5-thiadiazol-4-yl)-1,2,5,6-tetrahydro-1-methylpyridine; 3-(3-(4-Methylhexyloxy)-1,2,5-thiadiazol-4-yl)-1,2,5,6-tetrahydro-1-methylpyridine; 3-(3-(1-Methylhexyloxy)-1,2,5-thiadiazol-4-yl)-1,2,5,6-tetrahydro-1-methylpyridine; 3-(3-(3-Methylpentyloxy)-1,2,5-thiadiazol-4-yl)-1,2,5,6-tetrahydro-1-methylpyridine; 3-(3-Isoheptyloxy-1,2,5-thiadiazol-4-yl)-1,2,5,6-tetrahydro-1-methylpyridine; 3-(3-Isohexyloxy-1,2,5-thiadiazol-4-yl)-1,2,5,6-tetrahydro-1-methylpyridine; 3-(3-Hexyloxy-1,2,5-thiadiazol-4-yl)-1,2,5,6-tetrahydro-1-ethylpyridine; 3-(3-Ethoxy-1,2,5-thiadiazol-4-yl)-1,2,5,6-tetrahydropyridine; 3-(3-Cyclopropylmethoxy-1.,2,5-thiadiazol-4-yl)-1,2,5,6-tetrahydro-1-methylpyridine; 1,2,5,6-Tetrahydro-3-(3-methoxyethoxy-1,2,5-thiadiazol-4-yl)-1-methylpyridine; 3-(3-(2-(2-Methoxyethoxy)ethoxy)-1,2,5-thiadiazol-4-yl)-1,2,5,6-tetrahydro-1-methylpyridine; 3-(3-(3-Ethoxy-1-propoxy)-1,2,5-thiadiazol-4-yl)-1,2,5,6-tetrahydro-1-methy 1-pyridine; 3-(3-(2-Ethoxyethoxy)-1,2,5-thiadiazol-4-yl)-1,2,5,6-tetrahydro-1-methylpyridine; 3-(3-(2-Butoxyethoxy)-1,2,5-thiadiazol-4-yl)-1,2,5,6-tetrahydro-1-methylpyridine; 3-(3-(2-(2-Butoxyethoxy)ethoxy)-1,2,5-thiadiazol-4-yl)-1,2,5,6-tetrahydro-1-methylpyridine; 1,2,5,6-Tetrahydro-1-methyl-3-(3-(6,6,6-trifluorohexyloxy)-1,2,5-thiadiazol-4-yl)pyridine; 1,2,5,6-Tetrahydro-1-methyl-3-(3-(3-(4-methoxyphenyl)-1-propoxy)-1,2,5-thiadiazol-4-yl)pyridine; 1,2,5,6-Tetrahydro-1-methyl-3-(3-(2-(4-methoxyphenyl)-1-ethoxy)-1,2,5-thiadiazol-4-yl)pyridine; 1,2,5,6-Tetrahydro-1-methyl-3-(3-(3-hydroxy-1-propoxy)-1,2,5-thiadiazol-4-yl)pyridine; 1,2,5,6-Tetrahydro-1-methyl-3-(3-(2-phenyl-1-ethoxy)-1,2,5-thiadiazol-4-yl)pyridine; 1,2,5,6-Tetrahydro-1-methyl-3-(3-(3-hydroxy-1-hexyloxy)-1,2,5-thiadiazol-4-yl)pyridine; 1,2,5,6-Tetrahydro-1-methyl-3-(3-(3-phenyl-1-propoxy)-1,2,5-thiadiazol-4-yl)pyridine; 1,2,5,6-Tetrahydro-1-methyl-3-(3-(6-acetamido-1-hexyloxy)-1,2,5-thiadiazol-4-yl)pyridine; 1,2,5,6-Tetrahydro-1-methyl-3-(3-(2-acetamido-1-ethoxy)-1,2,5-thiadiazol-4-yl)pyridine; 1,2,5,6-Tetrahydro-1-methyl-3-(3-(2-propionamido-1-ethoxy)-1,2,5-thiadiazol-4-yl)pyridine; 1,2,5,6-Tetrahydro-1-methyl-3-(3-(2-benzylthio-1-ethoxy)-1,2,5-thiadiazol-4-yl)pyridine; 1,2,5,6-Tetrahydro-1-methyl-3-(3-(2-ureido-1-ethoxy)-1,2,5-thiadiazol-4-yl) pyridine; 3-(3-(4-Fluorophenylpropoxy)-1,2,5-thiadiazol-4-yl)-1,2,5,6-tetrahydro-1-methylpyridine; 3-(3-(4-Chlorophenylpropoxy)-1,2,5-thiadiazol-4-yl)-1,2,5,6-tetrahydro-1-methylpyridine; 3-(3-(3-Methylphenylpropoxy)-1,2,5-thiadiazol-4-yl)-1,2,5,6-tetrahydro-1-methylpyridine; 3-(3-(4-Methylphenylpropoxy)-1,2,5-thiadiazol-4-yl)-1,2,5,6-tetrahydro-1-methylpyridine; 3-(3-Phenylbutoxy-1,2,5-thiadiazol-4-yl)-1,2,5,6-tetrahydro-1-methylpyridine; 3-(3-(2-Methylphenylpropoxy)-1,2,5-thiadiazol-4-yl)-1,2,5,6-tetrahydro-1-methylpyridine; 3-(3-(2,5-Dimethylphenylpropoxy)-1,2,5-thiadiazol-4-yl)-1,2,5,6-tetrahydro-1-methylpyridine; 3-(3-(3,4-Dichlorophenylpropoxy)-1,2,5-thiadiazol-4-yl)-1,2,5,6-tetrahydro-1-methylpyridine; 3-(3-(4-Methylphenylpropoxy)--1,2,5-thiadiazol-4-yl)-1,2,5,6-tetrahydro-1methylpyridine; 3-(3-(4-Cyclohexylbutoxy)-1,2,5-thiadiazol-4-yl)-1,2,5,6-tetrahydro-1-methy-1-pyridine; 3-(3-(5-Hydroxyhexyloxy)-1,2,5-thiadiazol-4-yl)-1,2,5,6-tetrahydro-1-methylpyridine; 3-(3-(5-Oxyhexyloxy)-1,2,5-thiadiazol-4-yl)-1,2,5,6-tetrahydro-1-methylpyridine; 3-(3-(3-Cyclohexenylmethoxy)-1,2,5-thiadiazol-4-yl)-1,2,5,6-tetrahydro-1-methylpyridine; 3-(3-Isobutylthioethoxy-1,2,5-thiadiazol-4-yl)-1,2,5,6-tetrahydro-1-methylpyridine; 3-(3-Cyclopropylpropoxy-1,2,5-thiadiazol-4-yl)-1,2,5,6-tetrahydro-1-methylpyridine; 3-(3-(2-Methylcyclopropylmethoxy)-1,2,5-thiadiazol-4-yl)-1,2,5,6-tetrahydro-1-methylpyridine; 3-(3-Cyclopentylpropyloxy-1,2,5-thiadiazol-4-yl)-1,2,5,6-tetrahydro-1-methylpyridine; 3-(3-(4,4,4-Trifluorobutoxy)-1,2,5-thiadiazol-4-yl)-1,2,5,6-tetrahydro-1methylpyridine; 3-(3-(6,6,6-Trifluorohexyloxy)-1,2,5-thiadiazol-4-yl)-1,2,5,6-tetrahydro-1-methylpyridine; 3-(3-(3-Cyclobutylpropoxy)-1,2,5-thiadiazol-4-yl)-1,2,5,6-tetrahydro-1-methylpyridine; 3-(3-Isopropoxyethoxy-1,2,5-thiadiazol-4-yl)-1,2,5,6-tetrahydro-1-methylpyridine; 3-(3-(2,2,2-Trifluoroethoxy)-1,2,5-thiadiazol-4-yl)-1,2,5,6-tetrahydro-1-methylpyridine; 3-(3-(2-Chlorophenylpropoxy-1,2,5-thiadiazol-4-yl)-1,2,5,6-tetrahydro-1-methylpyridine; 3-(3-(3-Cyclohexylpropoxy)-1,2,5-thiadiazol-4-yl)-1,2,5,6-tetrahydro-1-methylpyridine; 3-(3-(2-Cyclohexylethoxy)-1,2,5-thiadiazol-4-yl)-1,2,5,6-tetrahydro-1-methylpyridine; 3-(3-(2,2,2-Trifluoroethoxy)-1,2,5-thiadiazol-4-yl)-1,2,5,6-tetrahydropyridine; 3-(3-(3-Carboxypropoxy)-1,2,5-thiadiazol-4-yl)-1,2,5,6-tetrahydro-1-methylpyridine; 3-(3-Benzyloxy-1,2,5-thiadiazol-4-yl)-1,2,5,6-tetrahydro-1-methylpyridine; 3-(3-(4-Ethylbenzyloxy)-1,2,5-thiadiazol-4-yl)-1,2,5,6-tetrahydro-1-methylpyridine; 3-(3-(4-Butylbenzyloxy)-1,2,5-thiadiazol-4-yl)-1,2,5,6-tetrahydro-1-methylpyridine; 3-(3-Propylthio-1,2,5-thiadiazol-4-yl)-1,2,5,6-tetrahydro-1-methylpyridine; 3-(3-Butylthio-1,2,5-thiadiazol-4-yl)-1,2,5,6-tetrahydro-1-methylpyridine; 3-(3-Methylthio-1,2,5-thiadiazol-4-yl)-1,2,5,6-tetrahydro-1-methylpyridine; 3-(3-Octylthio-1,2,5-thiadiazol-4-yl)-1,2,5,6-tetrahydro-1-methylpyridine; 3-(3-Ethylthio-1,2,5-thiadiazol-4-yl)-1,2,5,6-tetrahydro-1-methylpyridine; 3-(3-Pentylthio-1,2,5-thiadiazol-4-yl)-1,2,5,6-tetrahydro-1-methylpyridine; 3-(3-Hexylthio-1,2,5-thiadiazol-4-yl)-1,2,5,6-tetrahydro-1-methylpyridine; 3-(3-Hexylthio-1,2,5-thiadiazol-4-yl)-1,2,5,6-tetrahydro-1,6-dimethylpyridine; 3-(3-Pentylthio-1,2,5-thiadiazol-4-yl)-1,2,5,6-tetrahydro-1,6-dimethylpyridine; 3-(3-Butylthio-1,2,5-thiadiazol-4-yl)-1,2,5,6-tetrahydro-1,6-dimethylpyridine; 3-(3-Ethylthio-1,2,5-thiadiazol-4-yl)-1,2,5,6-tetrahydro-1,6-dimethylpyridine; 3-(3-(4-Pentynylthio)-1,2,5-thiadiazol-4-yl)-1,2,5,6-tetrahydro-1,6-dimethy-1-pyridine; 3-(3-Hexylthio-1,2,5-thiadiazol-4-yl)-1,2,5,6-tetrahydro-1-ethylpyridine; 3-(3-Ethylthio-1,2,5-thiadiazol-4-yl)-1,2,5,6-tetrahydro-1-ethylpyridine; 3-(3-Ethylthio-1,2,5-thiadiazol-4-yl)-1,2,5,6-tetrahydropyridine; 3-(3-Propylthio-1,2,5-thiadiazol-4-yl)-1,2,5,6-tetrahydropyridine; 3-(3-Butylthio-1,2,5-thiadiazol-4-yl)-1,2,5,6-tetrahydropyridine; 3-(3-Pentylthio-1,2,5-thiadiazol-4-yl)-1,2,5,6-tetrahydropyridine; 3-(3-Hexylthio-1,2,5-thiadiazol-4-yl-1,2,5,6-tetrahydropyridine; 3-(3-(4-Pentynylthio)-1,2,5-thiadiazol-4-yl)-1,2,5,6-tetrahydropyridine; 3-(3-Isohexylthio-1,2,5-thiadiazol-4-yl)-1,2,5,6-tetrahydro-1-methylpyridin e; cis-3-(3-(3-Hexenylthio)-1,2,5-thiadiazol-4-yl)-1,2,5,6-tetrahydro-1-methylpyridine; 3-(3-(5-Hexenylthio)-1,2,5-thiadiazol-4-yl)-1,2,5,6-tetrahydro-1-methylpyridine; 3-(3-Cyclopentylthio-1,2,5-thiadiazol-4-yl)-1,2,5,6-tetrahydro-1-methylpyridine; 3-(3-(4-Pentynylthio)-1,2,5-thiadiazol-4-yl)-1,2,5,6-tetrahydro-1-methylpyridine; 3-(3-Heptylthio-1,2,5-thiadiazol-4-yl)-1,2,5,6-tetrahydro-1-methylpyridine; 3-(3-(7-Octenylthio)-1,2,5-thiadiazol-4-yl)-1,2,5,6-tetrahydro-1-methylpyridine; 3-(3-(3-Butenylthio)-1,2,5-thiadiazol-4-yl)-1,2,5,6-tetrahydro-1-methylpyridine; 3-(3-(4-Pentenylthio)-1,2,5-thiadiazol-4-yl)-1,2,5,6-tetrahydro-1-methylpyridine; 3-(3-(5-Cyanopentylthio)-1,2,5-thiadiazol-4-yl)-1,2,5,6-tetrahydro-1-methylpyridine; 3-(3-(3-Chloropropylthio)-1,2,5-thiadiazol-4-yl)-1,2,5,6-tetrahydro-1-methylpyridine; 3-(3-(3-Cyanopropylthio)-1,2,5-thiadiazol-4-yl)-1,2,5,6-tetrahydro-1-methylpyridine; 3-(3-(3-Phenylpropylthio)-1,2,5-thiadiazol-4-yl)-1,2,5,6-tetrahydro-1-methylpyridine; 3-(3-(2-Phenoxyethylthio)-1,2,5-thiadiazol-4-yl)-1,2,5,6-tetrahydro-1-methylpyridine; 3-(3-(4-Cyanobutylthio)-1,2,5-thiadiazol-4-yl)-1,2,5,6-tetrahydro-1-methylpyridine; 3-(3-(8-Hydroxyoctylthio)-1,2,5-thiadiazol-4-yl)-1,2,5,6-tetrahydro-1-methylpyridine; 3-(3-(4-Chlorobutylthio)-1,2,5-thiadiazol-4-yl)-1,2,5,6-tetrahydro-1-methylpyrdine; 3-(3-(4,4-Bis-(4-fluorophenyl)-butylthio)-1,2,5-thiadiazol-4-yl)-1,2,5,6-tetrahydro-1-methylpyridine; 3-(3-(2-Phenylethylthio)-1,2,5-thiadiazol-4-yl)-1,2,5,6-tetrahydro-1-methylpyridine; 3-(3-(2-Benzoylethylthio)-1,2,5-thiadiazol-4-yl)-1,2,5,6-tetrahydro-1-methy-1-pyridine; 3-(3-(4,4,4-Trifluorobutylthio)-1,2,5-thiadiazol-4-yl)-1,2,5,6-tetrahydro-1-methylpyridine; 3-(3-(5,5,5-Trifluoropentylthio)-1,2,5-thiadiazol-4-yl)-1,2,5,6-tetrahydro-1-methylpyridine; 3-(3-(6,6,6-Trifluorohexylthio)-1,2,5-thiadiazol-4-yl)-1,2,5,6-tetrahydro-1-methylpyridine; 3-(3-Ethoxycarbonylpentylthio-1,2,5-thiadiazol-4-yl)-1,2,5,6-tetrahydro-1-methylpyridine; 3-(3-(4-Cyanobutylthio)-1,2,5-thiadiazol-4-yl)-1,2,5,6-tetrahydro-1,6-dimethylpyridine; 3-(3-(3-Phenylpropylthio)-1,2,5-thiadiazol-4-yl)-1,2,5,6-tetrahydro-1,6-dimethylpyridine; 3-(3-(2,2,2-Trifluoroethylthio)-1,2,5-thiadiazol-4-yl)-1,2,5,6-tetrahydropyridine; 3-(3-(2-Phenoxyethylthio)-1,2,5-thiadiazol-4-yl)-1,2,5,6-tetrahydropyridine; 3-(3-(2,2,2-Trifluoroethylthio)-1,2,5-thiadiazol-4-yl)-1,2,5,6-tetrahydro-1-methylpyridine; 3-(3-Ethoxycarbonylpropylthio-1,2,5-thiadiazol-4-yl)-1,2,5,6-tetrahydro-1-methylpyridine; 3-(3-(6-Hydroxyhexylthio)-1,2,5-thiadiazol-4-yl)-1,2,5,6-tetrahydro-1-methy-1-pyridine; 3-(3-(1-Cyclopropylmethylthio)-1,2,5-thiadiazol-4yl)-1,2,5,6-tetrahydro-1-methylpyridine; 3-(3-(2-Methoxyethylthio)-1,2,5-thiadiazol-4-yl)-1,2,5,6-tetrahydro-1-methy-1-pyridine; 3-(3-(2-(2-Ethoxymethoxy)-ethylthio)-1,2,5-thiadiazol-4-yl)-1,2,5,6-tetrahydro-1-methylpyridine; 3-(3-(2-Ethylbutylthio)-1,2,5-thiadiazol-4-yl)-1,2,5,6-tetrahydro-1-methylpyridine; 3-(3-Cyclohexylmethylthio-1,2,5-thiadiazol-4-yl)-1,2,5,6-tetrahydro-1-methy-1-pyridine; 3-(3-(3,3,3-Trifluoropropylthio)-1,2,5-thiadiazol-4-yl)-1,2,5,6-tetrahydro-1-methylpyridine; 3-(3-(1-Oxo-1-phenylpropylthio-1,2,5-thiadiazol-4-yl)-1,2,5,6-tetrahydro-1-methylpyridine; 3-(3-(4-Phenylthiobutylthio)-1,2,5-thiadiazol-4-yl)-1,2,5,6-tetrahydro-1-methylpyridine; 3-(3-Cyanomethylthio-1,2,5-thiadiazol-4-yl)-1,2,5,6-tetrahydro-1-methylpyridine; 3-(3-(6-Chlorohexylthio)-1,2,5-thiadiazol-4-yl)-1,2,5,6-tetrahydro-1-methylpyridine; 3-(3-(5-Chloropentylthio)-1,2,5-thiadiazol-4-yl)-1,2,5,6-tetrahydro-1-methy-1-pyridine; 3-(3-(3-Carboxypropylthio)-1,2,5-thiadiazol-4-yl)-1,2,5,6-tetrahydro-1-methylpyridine; 3-(3-(5-Carboxypentylthio)-1,2,5-thiadiazol-4-yl)-1,2,5,6-tetrahydro-1-methylpyridine; 3-(3-(5-Mercaptopentylthio)-1,2,5-thiadiazol-4-yl)-1,2,5,6-tetrahydro-1-methylpyridine; 3-(3-(6-Mercaptohexylthio)-1,2,5-thiadiazol-4-yl)-1,2,5,6-tetrahydro-1-methylpyridine; 3-(3-(4-Mercaptobutylthio)-1,2,5-thiadiazol-4-yl)-1,2,5,6-tetrahydro-1-methylpyridine; 3-(3-(4-Cyanobenzylthio)-1,2,5-thiadiazol-4-yl)-1,2,5,6-tetrahydro-1-methylpyridine; 3-(3-(4-Bromobenzylthio)-1,2,5-thiadiazol-4-yl)-1,2,5,6-tetrahydro-1-methylpyridine; 3-(3-(4-Methylbenzylthio)-1,2,5-thiadiazol-4-yl)-1,2,5,6-tetrahydro-1-methy-1-pyridine; 3-(3-Benzylthio-1,2,5-thiadiazol-4-yl)-1,2,5,6-tetrahydro-1-methylpyridine; 3-(3-(4-Cyanobenzylthio)-1,2,5-thiadiazol-4-yl)-1,2,5,6-tetrahydro-1,6-dimethylpyridine; 3-(3-Hexyloxy-1,2,5-oxadiazol-4-yl)-1,2,5,6-tetrahydro-1-methylpyridine; 3-(3-Butyloxy-1,2,5-oxadiazol-4-yl)-1,2,5,6-tetrahydro-1-methylpyridine; 3-(3-(3-Hexynyloxy)-1,2,5-oxadiazol-4-yl)-1,2,5,6-tetrahydro-1-methylpyridine; 3-(3-(3-Phenylpropylthio)-1,2,5-oxadiazol-4-yl)-1,2,5,6-tetrahydro 1-methylpyridine; 3-(3-(2-Phenoxyethylthio)-1,2,5-oxadiazol4-yl)-1,2,5,6-tetrahydro-1-methylpyridine; 3-(3-Pentylthio-1,2,5-oxadiazol-4-yl)-1,2,5,6-tetrahydro-1-methylpyridine; 3-(3-Hexylthio-1,2,5-oxadiazol-4-yl)-1,2,5,6-tetrahydro-1-methylpyridine; and 3-(3-(4-Pentynylthio)-1,2,5-oxadiazol-4yl)-1,2,5,6-tetrahydro-1-methylpyridine.

A more particularly preferred compound of formula II is xanomeline, 3-(4-hexyloxy-1,2,5-thiadiazol-3-yl)-1,2,5,6-tetrahydro-1-methylpyridine.

Preferred compounds of formula III are 2-[exo-(+/−)-3-[1-azabicyclo[3.2.1]octyloxy)]pyrazine, 3-butylthio-2-(1-azabicyclo[2.2.2]ocytl-3-oxy)]pyrazine, 3-butyloxy-2-[3-±-endo-(1-azabicyclo[2.2.1]heptyloxy)]pyrazine, 3-(2-butynyloxy)-2-[6-±-endo-(1-azabicyclo[3.2.1]octyloxy)pyrazine, 3-hexylthio-2-[6-±-exo-(2-azabicyclo[2.2.1]heptyloxy)]pyrazine, 3-(3-phenylpropynylthio)-2-[2-±-exo-(7-azabicyclo[2.2.1]heptyloxy)]pyrazine, 3-(2-methylthioethoxy)-2-[3-±-exo-(1-azabicyclo[3.2.1]octyloxy)]pyrazine, 3-propargyl-2-[4-(1-azabicyclo[2.2.1]heptyloxy)]pyrazine, and 3-cyclopropylmethylthio-2-[2-±-exo-(8-azabicyclo[3.2.1]octyloxy)]pyrazine

When the present invention is regarded in its broadest sense, the first component is either a typical or an atypical antipsychotic. Similarly, when the invention is regarded in its broadest sense, the second component compound is a compound which functions as a muscarinic agonist. It will be understood that while the use of a single typical antipsychotic or a single atypical antipsychotic as a first component compound is preferred, combinations of two or more antipsychotic may be used as a first component if necessary or desired. Similarly, while the use of a single muscarinic agonist as a second component compound is preferred, combinations of two or more muscarinic agonists may be used as a second component if necessary or desired.

While all combinations of first and second component compounds are useful and valuable, certain combinations are particularly valued and are preferred. In general, combinations and methods of treatment using an atypical antipsychotic as the first component are preferred. Particularly preferred combinations and methods of treatment using olanzapine as the first component are preferred. Furthermore, combinations and methods of treatment using xanomeline as the second component are preferred. Especially preferred are combinations and methods use olanzapine as the first component and xanomeline as the second component. It is especially preferred that when the first component is olanzapine, it will be the Form II olanzapine polymorph as described in U.S. Pat. No. 5,229,382. Form II olanzapine is characterized by x-ray powder diffraction pattern, of a well prepared sample, having an interplanar spacing at 10.2689. The x-ray diffraction pattern of Form II olanzapine can be obtained using a Siemens D5000 x-ray powder diffractometer having a copper K_{α} radiation source of wavelength, λ=1·541 Å.

It is further preferred that the Form II olanzapine polymorph will be administered as the substantially pure Form II olanzapine polymorph. As used herein “substantially pure” refers to Form II associated with less than about 5% Form I, preferably less than about 2% Form I, and more preferably less than about 1% Form I. Further, “substantially pure” Form II will contain less than about 0.5% related substances, wherein “related substances” refers to undesired chemical impurities or residual solvent or water. In particular, “substantially pure” Form II should contain less than about 0.05% content of acetonitrile, more preferably, less than about 0.005% content of acetonitrile. Additionally, the polymorph of the invention should contain less than 0.5% of associated water.

Though Form II olanzapine is preferred it will be understood that as used herein, the term “olanzapine” embraces all solvate and polymorphic forms except where specifically indicated.

As used in this application the following terms have the meanings indicated:

The term “halogen” means a fluoro, chloro, bromo, or iodo atom.

The term “C₁-₁₅-alkyl” represents a branched or linear alkyl group having from one to fifteen carbon atoms. Typical C₁-₁₅-alkyl groups include, but are not limited to, methyl, ethyl, n-propyl, iso-propyl, butyl, iso-butyl, sec-butyl, tert-butyl, pentyl, hexyl, heptyl, octyl, nonyl, decyl, and the like.

The term “C₁-₄-alkyl” represents a branched or linear alkyl group having from one to four carbon atoms. Typical C1-C4 alkyl groups include, but are not limited to, methyl, ethyl, n-propyl, iso-propyl, butyl, iso-butyl, sec-butyl, and tert-butyl.

The term “C₁₋₆-alkyl” represents a branched or linear alkyl group having from one to six carbon atoms. Typical C₁-₁₅ alkyl groups include, but are not limited to, methyl, ethyl, n-propyl, iso-propyl, butyl, isobutyl, sec-butyl, tert-butyl, pentyl, hexyl, and the like.

The term “C₂-₁₅-alkenyl” represents an branched or linear group having from two to fifteen carbon atoms and at least one double bond. Examples of such groups include, but are not limited to, 1-propenyl, 2-propenyl, 1-butenyl, hexenyl, pentenyl, hexenyl, heptenyl, octenyl, and the like.

The term “C₂₋₅-alkenyl” represents an branched or linear group having from two to five carbon atoms and at least one double bond. Examples of such groups include, but are not limited to, 1-propenyl, 2-propenyl, 1-butenyl, pentenyl, 2,2-dimethylpropenyl, and the like.

The term “C₂-C₁₅ alkynyl” refers to an unsaturated branched or linear group having from two to fifteen carbon atoms and at least one triple bond. Examples of such groups include, but are not limited to, 1-propynyl, 2-propynyl, 1-butynyl, 2-butynyl, 3-butynyl, 1-pentynyl, 1-hexynyl, 1-heptynyl, and the like.

The term “C₂₋₅-alkynyl” refers to an unsaturated branched or linear group having from two to five carbon atoms and at least one triple bond. Examples of such groups include, but are not limited to, 1-propynyl, 2-propynyl, 1-butynyl, 2-butynyl, 1-pentynyl, and the like.

The term “C₃-C₁₀ cycloalkyl” represents cyclopropyl, cyclobutyl, cyclopentyl, cyclohexyl, cycloheptyl, cyclooctyl, cyclononyl, and cyclodecyl.

The term “C₄₋₁₂-(cycloalkylalkyl)” refers to a C₃-C₁₀ cycloalkyl linked to a “C₁-₄-alkyl group in such a manner that the total number of carbon atoms in the group is between 4 and 12. Typical C₄₋₁₂-(cycloalkylalkyl) groups include cyclopropylmethyl, cyclopropylethyl, cyclopropylpropyl, cyclopropylbutyl, cyclobutylmethyl, cyclopentylmethyl, cyclohexylmethyl, cyclohexylethyl, cyclohexylmethyl, 3-cyclopentylpropyl, and the like.

The term “C₁-₄-alkoxyl” represents a branched or linear alkyl group having from one to four carbon atoms attached through and oxygen atom. Typical C₁-₄-alkoxy groups include, but are not limited to, methoxy, ethoxy, n-propoxy, iso-propoxy, butoxy, and the like.

The term “pharmaceutically acceptable salts” include the physiologically acceptable salts which are often used in pharmaceutical chemistry. Many such salts are described in Journal of Pharmaceutical Science, 66, 2 (1977). It will be understood by the skilled reader that some of the compounds used in the present invention are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them. It is also understood that the term “pharmaceutically acceptable salts” refers to acid addition and base addition salts. Thus, a pharmaceutically acceptable salt is formed from a pharmaceutically acceptable acid or a pharmaceutically-acceptable base as is well known in the art. Typical inorganic acids used to form acid addition salts include hydrochloric, hydrobromic, hydriodic, nitric, sulfuric, phosphoric, hypophosphoric, metaphosphoric, pyrophosphoric, and the like. Acid addition salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, isobutyrate, phenylbutyrate, -hydroxybutyrate, butyne-1,4-dicarboxylate, hexyne-1,4-dicarboxylate, caprate, caprylate, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, propiolate, propionate, phenylpropionate, salicylate, sebacate, succinate, suberate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like. Typical base addition salts include lithium, sodium, potassium, calcium, magnesium, aluminum, and the like.

The following examples are included to more specifically describe the preparation of the compounds used in the method of this invention. These examples are not intended to limit the present invention in any way.

The terms used in the following examples and preparations have their normal meanings unless otherwise designated. For example “° C.” refers to degrees Celsius; “mmol” refers to millimole or millimoles; “kg” refers to kilogram or kilograms; “g” refers to gram or grams; “mg” refers to milligram or milligrams; “mL” refers milliliter or milliliters; “L” refers to liter or liters; “brine” refers to a saturated aqueous sodium chloride solution; “min” refers to minute; “h” refers to hours, etc.

### EXAMPLE 1

Synthesis of Xanomeline

To a solution of sulfurmonochloride (2.4 ml, 30 mmol) in N,N-dimethylformamide (5 ml) was slowly added alpha-aminoalpha(3-pyridyl)acetonitrile (Archive der Pharmazie 289 (4) (1956)) (1.70 g, 10 mmol). The reaction mixture was stirred at room temperature for 18 h. Water (20 ml) was added and the aqueous phase was extracted with ether and the ether phase discharged. A 50% potassium hydroxide solution was added to the aqueous phase to pH>9. The aqueous phase was extracted several times with ether and the ether phases were dried and evaporated. The residue was purified by column chromatography (SiO.sub.2, eluent:ethyl acetate/methylene chloride (1:1)). 3-(3-Chloro-1,2,5-thiadiazol-4-yl)pyridine was collected in 45% (880 mg) yield. M⁺: 197.

To a solution of sodium (230 mg, 10 mmol) in 1-hexanol (15 ml) was added 3-(3-chloro-1,2,5-thiadiazol-4-yl)pyridine (490 mg, 2.5 mmol). The mixture was stirred at 50° C. for 2 h and evaporated. The residue was dissolved in water and extracted with ether. The combined organic phases were dried and evaporated to give 3-(3-Hexyloxy-1,2,5-thiadiazol-4-yl)pyridine.

A mixture of methyl iodide (0.5 ml, 7.5 mmol) and 3-(3-hexyloxy-1,2,5-thiadiazol-4-yl)pyridine (658 mg, 2.5 mmol) in acetone (5 ml) was stirred at room temperature for 18 h. 3-(3-Hexyloxy-1,2,5-thiadiazol-4-yl)-1-methylpyridinium iodide precipitated from the solution and was collected by filtration to yield 0.81 g (80%).

Sodium borohydride (230 mg, 6 mmol) was added to a solution of 3-(3-hexyloxy-1,2,5-thiadiazol-4-yl)-1-methylpyridinium iodide (810 mg, 2 mmol) in ethanol (99.9%, 20 ml) and the reaction mixture was stirred at room temperature for 1 h. After evaporation the residue was dissolved in water and extracted with ethyl acetate. The dried organic phases were evaporated and the residue purified by column chromatography (SiO₂, eluent:ethyl acetate/methanol (4:1)). 3-(3-Hexyloxy-1,2,5-thiadiazol-4-yl)-1,2,5,6-tetrahydro-1-methylpyridine oxalate was crystallized as the oxalate salt from acetone to yield 350 mg. (M.p. 148° C.; M⁺: 281;).

### EXAMPLE 2

Technical Grade Olanzapine

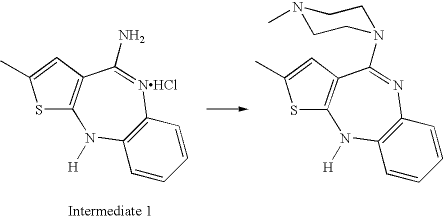

In a suitable three neck flask the following was added:

| | |
|---|---|
| Dimethylsulfoxide (analytical): | 6 volumes |
| Intermediate 1: | 75 g |
| N-Methylpiperazine (reagent): | 6 equivalents |

Intermediate 1 can be prepared using methods known to the skilled artisan. For example, the preparation of the Intermediate 1 is taught in U.S. Pat. No. 5,229,382.

A sub-surface nitrogen sparge line was added to remove the ammonia formed during the reaction. The reaction was heated to 120° C. and maintained at that temperature throughout the duration of the reaction. The reactions were followed by HPLC until 5% of the intermediate 1 was left unreacted. After the reaction was complete, the mixture was allowed to cool slowly to 20° C. (about 2 hours). The reaction mixture was then transferred to an appropriate three neck round bottom flask and water bath. To this solution with agitation was added 10 volumes reagent grade methanol and the reaction was stirred at 20° C. for 30 minutes. Three volumes of water was added slowly over about 30 minutes. The reaction slurry was cooled to zero to 5° C. and stirred for 30 minutes. The product was filtered and the wet cake was washed with chilled methanol. The wet cake was dried in vacuo at 45° C. overnight. The product was identified as technical olanzapine. Yield: 76.7%; Potency: 98.1%

### EXAMPLE 3

Form II Olanzapine Polymorph

A 270 g sample of technical grade 2-methyl-4-(4-methyl-1-piperazinyl)-10H-thieno[2,3-b][1,5]benzodiazepine was suspended in anhydrous ethyl acetate (2.7 L). The mixture was heated to 76° C. and maintained at 76° C. for 30 minutes. The mixture was allowed to cool to 25° C. The resulting product was isolated using vacuum filtration. The product was identified as Form II using x-ray powder analysis.

Yield: 197 g.

The process described above for preparing Form II provides a pharmaceutically elegant product having potency>97%, total related substances<0.5% and an isolated yield of >73%.

According to the present invention the combination is usually administered in the form of pharmaceutical compositions. The adjunctive therapy of the present invention is carried out by administering a first component together with the second component in any manner which provides effective levels of the compounds in the body at the same time. Oral administration of the adjunctive combination is preferred. They may be administered together, in a single dosage form, or may be administered separately.

Oral administration is not the only route or even the only preferred route. For example, transdermal administration may be very desirable for patients who are forgetful or petulant about taking oral medicine. One of the drugs may be administered by one route, such as oral, and the others may be administered by the transdermal, percutaneous, intravenous, intramuscular, intranasal or intrarectal route, in particular circumstances. The route of administration may be varied in any way, limited by the physical properties of the drugs and the convenience of the patient and the caregiver.

The adjunctive combination may be administered as a single pharmaceutical composition, and so pharmaceutical compositions incorporating both compounds are important embodiments of the present invention. Such compositions may take any physical form which is pharmaceutically acceptable, but orally usable pharmaceutical compositions are particularly preferred. Such adjunctive pharmaceutical compositions contain an effective amount of each of the compounds, which effective amount is related to the daily dose of the compounds to be administered. Each adjunctive dosage unit may contain the daily doses of all compounds, or may contain a fraction of the daily doses, such as one-third or one-half of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

The inert ingredients and manner of formulation of the adjunctive pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compounds, however, is best defined as the effective amount, that is, the amount of each compound which provides the desired dose to the patient in need of such treatment. The activity of the adjunctive combinations do not depend on the nature of the composition, so the compositions are chosen and formulated solely for convenience and economy. Any of the combinations may be formulated in any desired form of composition. Some discussion of different compositions will be provided, followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidone and the like. Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Tablets are often coated with sugar as a flavor and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with innumerable pores through which the drugs are pumped by osmotic action. Transdermal formulations for administration of xanomeline are described in U.S. Pat. No. 5,980,933.

The following typical formula are provided for the interest and information of the pharmaceutical scientist.

### Formulation 1

Hard gelatin capsules are prepared using the following ingredients:

| Quantity | |
|---|---|
| (mg/capsule) | |
| Olanzapine | 25 mg |
| Xanomeline | 80 |
| Starch, dried | 150 |
| Magnesium stearate | 10 |
| Total | 265 mg |

### Formulation 2

A tablet is prepared using the ingredients below:

| Quantity | |
|---|---|
| (mg/tablet) | |
| Olanzapine | 10 |
| Fluoxetine, racemic, hydrochloride | 80 |
| Cellulose, microcrystalline | 275 |
| Silicon dioxide, fumed | 10 |
| Stearic acid | 5 |
| Total | 375 mg |

The components are blended and compressed to form tablets.

Conditioned Avoidance Behavior in Rats:

In a conditioned avoidance test, animals learn to respond during a conditioned stimulus in order to avoid mild shock presentation. A response during the conditioned stimulus is termed an avoidance response, a response during shock is termed an escape response; a response failure is when the animal fails to respond during either the conditioned stimulus or the shock presentation and is indicative of motor impairment. Animals rapidly learn to avoid 99% of the time. Antipsychotic drugs decrease the percentage of avoidance responses without interfering with the ability of the animal to respond since the animals do emit escape responses. The percentage of response failures is considered a measure of motor impairment.

Procedure:

Rats were required to press a response lever in an experimental chamber in order to avoid or escape foot-shock. Each experimental session consisted of 50 trials. During each trial, the chamber was illuminated and a tone presented for a maximum of 10 sec. A response during the tone immediately terminated the tone and the houselight, ending the trial. In the absence of a response during the tone alone, tone+foot-shock (2.0 mA) was presented for a maximum of 10 sec. A response during shock presentation immediately terminated the shock, the tone and the houselights ending the trial.

The synergistic effects of the present combinations can be demonstrated by administering an inactive or relatively inactive dose of one component (such as the selected muscarinic agonist) and various doses of the other component. If the effects are simply additive the results will be additive. If the effects of are synergistic, then the dose of second component required to produce full efficacy will be decreased. In particular, if the dose of both components are inactive when administered alone, but produce full efficacy when given in combination, then the combination is synergistic.

Results for representative combinations of the present invention are given in the table below.

| | | | | | % |
|---|---|---|---|---|---|
| | | | | % Avoidance | Response |
| First | | Second | | Responses ± | Failures ± |
| Component | Dose | Component | Dose | SEM | SEM |
| — | — | Xanomeline | 0 | 98 ± 1 | 0 ± 0 |
| — | — | | 1 | 99 ± 0 | 0 ± 0 |
| — | — | | 3 | 93 ± 3 | 0 ± 0 |
| — | — | | 10 | 63 ± 18 | 1 ± 0 |
| — | — | | 17.5 | 11 ± 4 | 2 ± 1 |
| — | — | | 30 | 2 ± 1 | 21 ± 14 |
| Olanzapine | 0 | — | — | 100 ± 0 | 0 ± 0 |
| | 1 | — | — | 98 ± 1 | 0 ± 0 |
| | 3 | — | — | 59 ± 16 | 1 ± 1 |
| | 10 | — | — | 2 ± 1 | 5 ± 3 |
| Olanzapine | 0 | Xanomeline | 10 | 87 ± 9 | 0 ± 0 |
| | 0.01 | | 10 | 66 ± 11 | 1 ± 1 |
| | 0.03 | | 10 | 47 ± 12 | 0 ± 0 |
| | 0.1 | | 10 | 56 ± 6 | 0 ± 0 |
| | 0.3 | | 10 | 53 ± 14 | 17 ± 10 |
| | 1.0 | | 10 | 9 ± 6 | 6 ± 5 |
| Haloperidol | 0 | — | — | 99.0 ± 1.0 | 0 ± 0 |
| | 0.03 | — | — | 98.0 ± 1.0 | 1 ± 0 |
| | 0.1 | — | — | 59.0 ± 13.0 | 2 ± 1 |
| | 0.3 | — | — | 10.0 ± 2.0 | 25 ± 12 |
| Haloperidol | 0.001 | Xanomeline | 10 | 70 ± 12 | 5 ± 3 |
| | 0.003 | | 10 | 45 ± 13 | 14 ± 11 |
| | 0.01 | | 10 | 57 ± 6 | 1 ± 0 |
| | 0.03 | | 10 | 27 ± 8 | 8 ± 6 |
| Chlor- | 0 | — | — | 99 ± 1 | 0 ± 0 |
| promazine | 1.25 | — | — | 99 ± 1 | 0 ± 0 |
| | 2.5 | — | — | 96 ± 4 | 0 ± 0 |
| | 5 | — | — | 27 ± 12 | 7 ± 5 |
| | 10 | — | — | 10 ± 4 | 16 ± 8 |
| Chlor | 0 | Xanomeline | 10 | 56 ± 13 | 0 ± 0 |
| promazine | 0.16 | | 10 | 56 ± 11 | 1 ± 1 |
| | 0.32 | | 10 | 46 ± 22 | 19 ± 19 |
| | 0.625 | | 10 | 30 ± 17 | 0 ± 0 |
| | 1.25 | | 10 | 20 ± 10 | 8 ± 7 |
| | 2.5 | | 10 | 22 ± 7 | 1 ± 0 |
| Fluphenazine | 0 | — | — | 99 ± 1 | 0 ± 0 |
| | 0.1 | — | — | 100 ± 0 | 0 ± 0 |
| | 0.3 | — | — | 67 ± 9 | 2 ± 1 |
| | 1.0 | — | — | 33 ± 6 | 28 ± 9 |
| Fluphenazine | 0 | Xanomeline | 10 | 87 ± 9 | 0 ± 0 |
| | 0.01 | | 10 | 77 ± 12 | 1 ± 0 |
| | 0.03 | | 10 | 52 ± 12 | 3 ± 2 |
| | 0.1 | | 10 | 66 ± 11 | 0 ± 0 |
| | 0.3 | | 10 | 33 ± 16 | 6 ± 6 |
| Risperidone | 0 | — | — | 100 ± 0 | 0 ± 0 |
| | 0.03 | — | — | 100 ± 0 | 0 ± 0 |
| | 0.1 | — | — | 91 ± 5 | 0 ± 0 |
| | 0.3 | — | — | 37 ± 11 | 2 ± 2 |
| | 1.0 | — | — | 2 ± 1 | 38 ± 9 |
| Risperidone | 0 | Xanomeline | 10 | 72 ± 13 | 0 ± 0 |
| | 0.001 | | 10 | 48 ± 8 | 0 ± 0 |
| | 0.003 | | 10 | 48 ± 16 | 2 ± 1 |
| | 0.01 | | 10 | 23 ± 14 | 0 ± 0 |
| | 0.03 | | 10 | 8 ± 2 | 7 ± 4 |
| — | — | Xanomeline | 0 | 100 ± 0 | 0 ± 0 |
| — | — | | 1 | 98 ± 2 | 0 ± 0 |
| — | — | | 3 | 93 ± 3 | 0 ± 0 |
| — | — | | 10 | 44 ± 18 | 2 ± 1 |
| — | — | | 30 | 9 ± 12 | 12 ± 10 |
| — | — | Xanomeline | 0 | 100 ± 0 | 0 ± 0 |
| — | — | | 3 | 99 ± 1 | 0 ± 0 |
| — | — | | 10 | 78 ± 12 | 1 ± 0 |
| — | — | | 30 | 43 ± 13 | 9 ± 7 |

We claim:

1. A method for treating a patient suffering from or susceptible to psychosis, comprising administering to the patient an effective amount a first component which is a typical antipsychotic or an atypical antipsychotic and an effective amount of a second component which is a muscarinic agonist.

2. A method of claim 1 where the first component is olanzapine and the second component is xanomeline.

3. A method of claim 1 wherein the patient is suffering from schizophrenia.

4. A method of claim 2 wherein the patient is suffering from schizophrenia.

5. A method of treating a patient suffering or susceptible to Alzheimer's disease, comprising administering to the patient an effective amount a first component which is a typical antipsychotic or an atypical antipsychotic and an effective amount of a second component which is a muscarinic agonist.

6. A pharmaceutical composition comprising an effective amount a first component which is a typical antipsychotic or an atypical antipsychotic and a second component which is a muscarinic agonist.

7. The use of a first component which is a typical antipsychotic or an atypical antipsychotic and an effective amount of a second component which is a muscarinic agonist for the manufacture of a medicament for treating psychosis.

8. A use of claim 7 where the first component is olanzapine and the second component is xanomeline.

9. A use of claim 7 wherein the patient is suffering from schizophrenia.

10. A use of claim 8 wherein the patient is suffering from schizophrenia.

11. The use of a first component which is a typical antipsychotic or an atypical antipsychotic and an effective amount of a second component which is a muscarinic agonist for the manufacture of a medicament for treating Alzheimer's disease.
